(19) United States
(12) Patent Application Publication
(10) Pub. No.: US 2006/0224098 A1
(43) Pub. Date: Oct. 5, 2006

(54) Cranial treatment apparatus

(75) Inventors: Tsuyoshi Uetake, Tokyo (JP); Yasushi Sugawara, Kawachi-gun (JP)

Correspondence Address: DARBY & DARBY P.C., P. O. BOX 5257, NEW YORK, NY, 10150-5257, US

(73) Assignee: KEISEI MEDICAL INDUSTRIAL CO., LTD., Tokyo (JP)

(21) Appl. No.: 11/255,241

(22) Filed: Oct. 21, 2005

Related U.S. Application Data
(60) Provisional application No. 60/668,893, filed on Apr. 5, 2005.

(51) Int. Cl. A61F 13/12

(52) U.S. Cl. 602/74

(57) ABSTRACT

A cranial treatment apparatus has a head portion arrangement body for arranging around a head, a retainer member retained at each cranial piece being tensed by a tension maintenance member for maintaining a tensed condition so that cranial pieces are tensed in a direction away from each other. The retainer member is provided in the head portion arrangement body.

### FIELD OF THE INVENTION

The present invention relates to a cranial treatment apparatus.

### BACKGROUND ART

A cranium of an infant who are growing up is divided into a plurality of portions along its cranial suture. The cranium is also glowing up corresponding to the growth (swell-out) of its brain. The confronting edge portions of the cranial suture which are separated from each other due to the growth of the brain expand in a direction in which the confronting edge portions are agglutinated with each other (stick each other). Accordingly, the cranium becomes swelled. At the time when the growth of the brain is completed, this cranial suture is agglutinated.

By the way, there is a disease of cranial early-stage agglutination in which a part of or all the cranial suture is agglutinated before the growth of the brain is completed, the growth of the brain is advanced under the condition that this cranial suture is agglutinated so that the brain is compressed and furthermore, the cranium is deformed.

So far, the following apparatus (hereinafter referred to as a related art) is proposed as a cranial treatment apparatus for treating the disease of cranial early-stage agglutination.

As shown in FIG. 23, the related art is comprised of a fastening member 34 attached to each cranial piece 33 separated from the agglutinated portion of the cranial piece 33 and a screw rod 35 inserted into an insertion portion 34a of the fastening member 34 attached to each cranial piece 33 and kept under a beam condition between the fastening members 34. This screw rod 35 is threadedly moved so that the fastening members 34 attached to the cranial pieces 33 are separated in a direction indicated by an arrow a from each other to thereby prevent the cranial pieces 33 from being agglutinated from each other. While gradually increasing a distance between the cranial pieces 33, the cranium is expanded without any hindrance against the growth of the brain.

This related art is extensively applied to a cranial treatment (i.e., bone extension treatment) such as a treatment of the disease of early-stage agglutination, a treatment of a malformation, a treatment of broken bones or the like.

The Tasks to be Solved by the Invention

However, the present applicants have tried about the related art in various ways and have found the problem in which, due to the structure of the related art in which the interval between the cranial pieces 33a is adjusted simply by moving the retainer members 34 relative to the linear screw rod 35 as described above, the cranial pieces 33 are separated from each other in the circumferential direction of the cranium (in the direction indicated by the arrow a in FIG. 23) and the cranial pieces 33 could not move in the radial direction of the cranium as a result of which the cranium pieces 33 could not be corrected well in accordance with the development of the brain (it is impossible to correct the cranium to be the cranium having a spherical round shape).

The present applicants have repetitively studied and developed in view of the foregoing points as a result of which the applicant have developed a novel cranium treatment apparatus exhibiting the effects that have not been attain by the conventional art.

Means for Solving the Tasks

The essence of the present invention will now be described with reference to the accompanying drawings.

The invention relates to a cranial treatment apparatus having a head portion arrangement body 2 arranged around a head 1, characterized in that a retainer member 4 retained at each cranial piece 3 is tensed by a tension maintenance member 16 for maintaining a tensed condition that cranial pieces 3 are tensed in a direction away from each other is provided in the head portion arrangement body 2.

Also, the cranial treatment apparatus according to claim 1 is further characterized in that the direction in which said tension maintenance member 16 tenses is in a radial direction of the head 1.

Also, the cranial treatment apparatus according to claim 1 is further characterized in that said tension maintenance member 16 is adapted to tense the retainer member 4 and maintain the tensed condition.

Also, the cranial treatment apparatus according to claim 2 is further characterized in that said tension maintenance member 16 is adapted to tense the retainer member 4 and maintain the tensed condition.

Also, the cranial treatment apparatus according to any one of claims 1 to 4 is further characterized in that said retainer member 4 and said tension maintenance member 16 are coupled with each other by a line-like member 5 so that the retainer member 4 is tensed by the line-like member 5 and the tensed condition is kept by the tension maintenance member 16.

Also, the cranial treatment apparatus according to claim 5 is further characterized in that a fastening member 32 is detachably provided in said head portion arrangement body 2 for fixing the line-like member 5 that has been removed from the tension maintenance member 16 after the tensed condition completion of the line-like member 5 by said tension maintenance member 16 and a projecting amount from a surface of said head portion arrangement body 2 of said fastening member 32 is set to be smaller than a projecting amount from a surface of the head portion arrangement body 2 of said tension maintenance member 16.

Also, the cranial treatment apparatus according to claim 6 is further characterized in that a hole portion 32a for allowing the line-like member 5 extending from the retainer member 4 to pass therethrough is provided in said fastening member 32.

Also, the cranial treatment apparatus according to any one of claims 1 to 4 is further characterized in that said retainer member 4 has an insertion portion 4a which is inserted into the cranial piece 3, a large diameter portion 4b which is larger in diameter than the insertion portion 4a and prevents the insertion portion 4a from entering into the cranial piece 3 is formed on a proximal end side of the insertion portion 4a, and a lower surface or of the side of the insertion portion 4a of the large diameter portion 4b is formed into a curved surface or a slant surface.

Also, the cranial treatment apparatus according to claim 5 is further characterized in that said retainer member 4 has an insertion portion 4a to be inserted into the cranial piece 3, a large diameter portion 4b that is larger in diameter than the insertion portion 4a for preventing the insertion portion 4a from entering into the cranial piece 3 is formed on the proximal end side of the insertion portion 4a, and the lower surface on the insertion portion 4a side of the large diameter portion 4b is formed into a curved surface or a slant surface.

Also, the cranial treatment apparatus according to claim 6 is further characterized in that said retainer member 4 has an insertion portion 4a to be inserted into the cranial piece 3, a large diameter portion 4b that is larger in diameter than the insertion portion 4a for preventing the insertion portion 4a from entering into the cranial piece 3 is formed on the proximal end side of the insertion portion 4a, and the lower surface on the insertion portion 4a side of the large diameter portion 4b is formed into a curved surface or a slant surface.

Also, the cranial treatment apparatus according to claim 7 is further characterized in that said retainer member 4 has an insertion portion 4a to be inserted into the cranial piece 3, a large diameter portion 4b that is larger in diameter than the insertion portion 4a for preventing the insertion portion 4a from entering into the cranial piece 3 is formed on the proximal end side of the insertion portion 4a, and the lower surface on the insertion portion 4a side of the large diameter portion 4b is formed into a curved surface or a slant surface.

Effects of the Invention

With such a structure of the invention as described above, it is possible to provide a novel cranial treatment apparatus that may perform a high quality treatment with the cranium kept under the very good condition after the treatment.

The Best Mode for Embodying the Invention

The best mode for embodying the invention which is considered as a preferable mode will now be described with reference to the accompanying drawings with the resultant effects in brief.

According to the invention, the head portion arrangement body 2 is arranged around the head 1, and the tensed condition in which the retainer member 4 retained at the cranial piece 3 is tensed by the tension maintenance member 16 provided in the head portion arrangement body 2 is maintained. This tensed condition is the tensed condition in which the cranial pieces 3 are separated away from each other. It is therefore possible to prevent the agglutination of the cranial pieces 3 with each other.

For example, in the case where the invention is applied to the treatment for the early stage agglutination of the cranium, since the condition that the respective cranial pieces 3 are tensed in a direction away from each other (in the radial direction of the cranium 1) is maintained, the agglutination of the cranium pieces 3 is prevented and furthermore, since the cranial pieces 3 is not tensed in the two-dimensional direction as in the related art but is tensed in the three-dimensional direction, the direction is identified with the direction of development of the brain (in the swelling direction). Accordingly, it is possible to form the shape of the cranial piece 3 after the treatment in a very good condition.

Also, after the completion of the tensed condition of the line-like member 5 by the tension maintenance member 16, the fastening member 32 for fixing the line-like member 5 removed from the tension maintenance member 16 is detachably provided to the above-described head portion arrangement body 2. Under the condition that the fastening member 32 is arranged on the head portion arrangement body 2, the projecting amount from the surface of the head portion arrangement body 2 is smaller than the projecting amount projecting from the head portion arrangement body 2 of the above-described tension maintenance member 16. In this case, (in which this treatment of the early-stage agglutination of the cranium has the extension work period to tense the cranial pieces 3 for keeping the tensed condition and the fixing work period for keeping and fixing the cranial pieces 3 to such an extent that the cranial pieces 3 are not returned back to the original position after this extension work period, and for the former case, the tension maintenance member 16 is used, whereas for the latter case, the fastening member 32 is used), if the tension maintenance member 16 projecting from the head portion arrangement member 2 is removed, there is no obstruction of projection projecting from the head portion arrangement body 2 (since the tension maintenance member 16 is used both for the extension work and the fixing work, the structure thereof is inevitably complicated to be enlarged). Thus, the treatment is well performed. For example, the treatment of the early-stage cranium agglutination is applied to the infant as described before. If a number of the tension maintenance members 16 project from the head portion arrangement body 2, for example, when the infant plays, there is a problem that the tension maintenance members 16 would hook at something. Unless there is such a projection of the tension maintenance member 16, there is no fear that such an accident happens. It is possible to perform the treatment in safety.

### BRIEF DESCRIPTION OF THE DRAWINGS

FIG. 1 is a perspective view showing the present embodiment.

FIG. 2 is an enlarged view of a primary part of the present embodiment.

FIG. 3 is a perspective view showing the primary part of the present embodiment.

FIG. 4 is a schematic operational view of the primary part of the present embodiment.

FIG. 5 is a schematic illustrative perspective view of the present embodiment.

FIG. 6 is an illustrative view of the primary part according to the present embodiment.

FIG. 7 is an illustrative view of the primary part according to the present embodiment.

FIG. 8 is a perspective view of the primary part according to the present embodiment.

FIG. 9 is a perspective view of the primary part according to the present embodiment.

FIG. 10 is a frontal view of the primary part according to the present embodiment.

FIG. 11 is a reference illustrative view of the primary part according to the present embodiment.

FIG. 12 is a reference illustrative view of the primary part according to the present embodiment.

FIG. 13 is an illustrative view of a retainer member 4 according to the present embodiment.

FIG. 14 is an illustrative view of a comparative structure of the retainer member 4 according to the present embodiment.

FIG. 15 is a perspective view of the primary part according to the present embodiment.

FIG. 16 is a perspective view of the primary part according to the present embodiment.

FIG. 17(a) to 17(c) are schematic illustrative views of the primary part according to the present embodiment.

FIG. 18 is an illustrative view of the used condition according to the present embodiment.

FIG. 19 is an illustrative view of the used condition according to the present embodiment.

FIG. 20 is an illustrative view of the used condition according to the present embodiment.

FIG. 21 is an illustrative view of the used condition according to the present embodiment.

FIG. 22 is an illustrative view of the used condition according to the present embodiment.

FIG. 23 is an illustrative view of the used condition according to the related art.

In the drawings:

Reference numeral 1 denotes a head.
2: head portion arrangement body
3: cranial piece
4: retainer portion
4a: insertion portion
4b: large diameter portion
5: line-like member
6: tension maintenance portion
32: fastening member
32a: hole portion

### EMBODIMENTS

An embodiment of the invention will now be described with reference to the accompanying drawings.

In the embodiment, there is provided a head portion arrangement body 2 to be arranged around a head portion 1. A tension maintenance member 16 for maintaining a retainer member 4 to be retained to a cranium piece 3 in a tensed condition in which the cranium pieces 3 are tensed in a direction in which the cranium pieces 3 are separated away from each other is provided in this head portion arrangement body 2.

Structural elements of this embodiment will now be described in more detail.

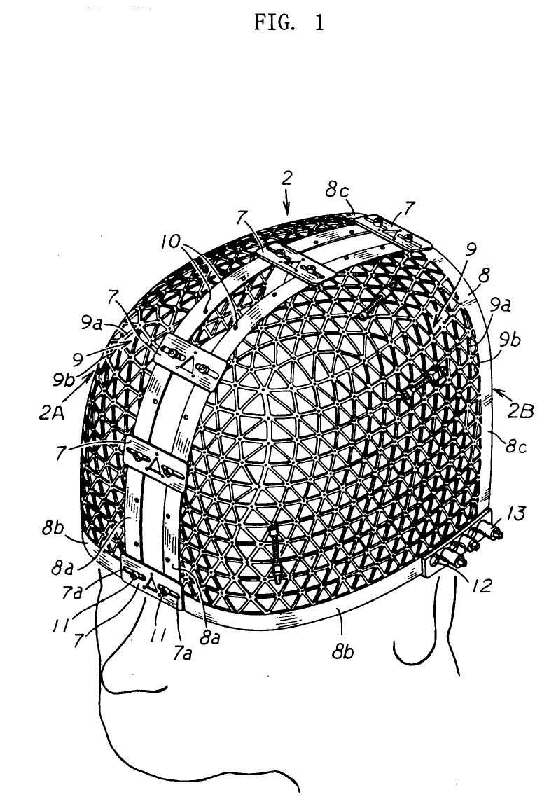

As shown in FIG. 1, the head portion arrangement body 2 is obtained by forming a suitable thin plate-like metal material into a curved shape (mesh-like or helmet-like shape) and by coupling a pair of right and left halves 2A and 2B with each other.

More specifically, each of the halves 2A and 2B is composed of a frame portion 8 composed of a central frame portion 8a, a lower frame portion 8b and a rear frame portion 8c and a beam portion 9 to be laid between the frame portions 8 as shown in FIG. 1, respectively.

A plurality of holes are formed in the central frame portion 8a at predetermined intervals. Also, the halves 2A and 2B are coupled with each other by a joint member 7.

Each joint member 7 is obtained by forming a suitable metal material into a plate-like shape. Oblong joint holes 7a through which retainer members 11 may be coupled at any desired positions are provided in right and left sides of the joint member 7. Accordingly, the halves 2A and 2B are separated, shifted and then fixed at any desired positions to each other by the joint members 7 so that a size of the head portion arrangement body 2 may fit the head 1.

A plurality (six) of holes 12 are arranged in parallel at rear positions of the lower frame portion 8b of the frame portion 8 and are adapted to serve as insertion holes 12 through which fastening rod members 13 for fixing the head portion arrangement body 2 (halves 2A and 2B) to the head 1 is caused to pass.

Each fastening rod member 13 is obtained by forming a suitable metal material into a rod-shape. A screw portion 13a which is to be threadedly coupled to a proximal end portion of a retainer member 14 retained at a side portion of the cranium to be described later is projectingly provided in a tip end portion of the fastening rod member 13. The latter is threadedly attached to the retainer member 14 under the condition that, the fastening rod member 13 is inserted through a through hole 12 provided in the head portion arrangement body 2 so that the head portion arrangement body 2 is fixed to the upper portion of the head 1 at a predetermined interval.

Incidentally, the retainer member 14 is retained at a retainer hole 3a formed in the cranium by using a boring tool 21 and a retainer portion forming tool 18 to be described later.

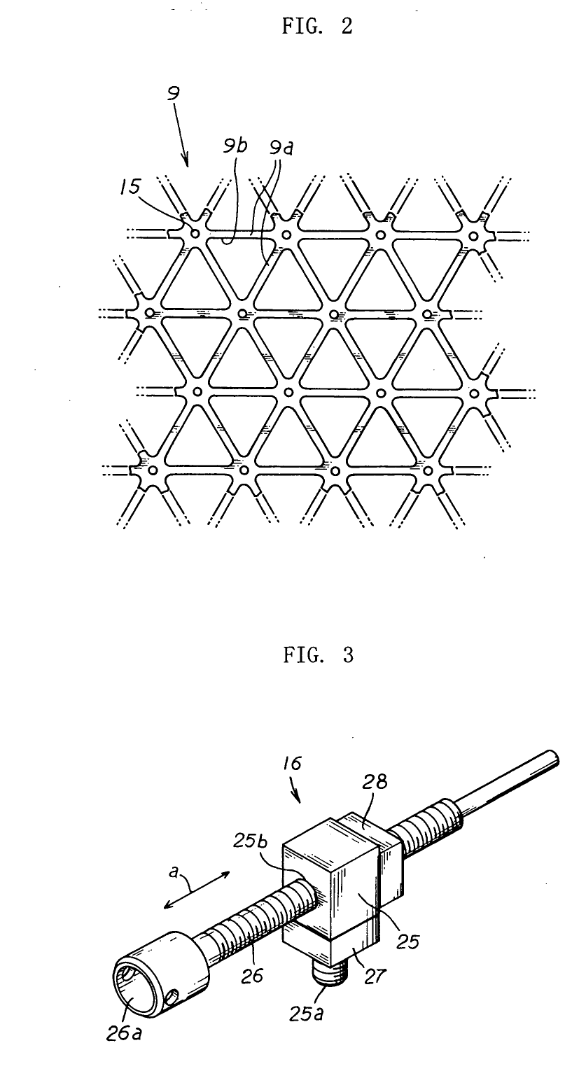

As shown in FIGS. 1 and 2, the beam portion 9 is formed into a mesh-like shape in which line-like members 9a are crossed with each other providing a number of through holes 9b on its surface. In the case of the embodiment shown in the drawings, each through hole 9b is formed into a triangular shape. The line-like members 5 coupled with the retainer members 4 to be described later through the through holes 9b may be arranged outside of the head portion arrangement body 2. Incidentally, the beam portion 9 is not limited to the line-like members 9a but may be strip-like members.

It is possible to take any size of the through holes 9b but it is better to take a large size of the through holes 9b as much as possible in view of a light weight, permeability and an easy-to-handle property of the line-like members 5. In addition, it is possible to propose to take a round shape or a rectangular shape as a shape of the through holes 9b but the triangular shape is suitable because the material may be taken as much as possible. Furthermore, in view of the mechanical strength and the mass production aspect, it is preferable to take the triangular shape.

Also, the beam portion 9 is provided with a hole 15 at each cross portion of the line-like members 9a. This hole 15 is formed into a screw hole 15 for threadedly engaging with a tension maintenance member 16 to be described later.

The tension maintenance member 16 is detachably provided to the head portion arrangement body 2.

As shown in FIG. 3, this tension maintenance member 16 is composed, in its lower portion, of a coupling body 25 projectingly provided with a screw portion 25a threadedly engaging with the screw hole 15 provided in the above-described head portion arrangement body 2 and a shifting member 26 to be threadedly engaged with the screw hole 25b passing through front and rear surfaces of the coupling member 25. Accordingly, the tension maintenance member 16 is detachable at any position of the head portion arrangement body 2.

In this shifting member 26, a sleeve-like member 26a is rotatably fitted at a port ion projecting from a front portion of the coupling member 25 at the front tip portion thereof. This sleeve-like member 26a is formed into the coupling portion 26a for coupling with the line-like member 5 (wire) coupled to the retainer member 4 to be described later.

Accordingly, when the shifting member 26 is threadedly moved, the coupling port ion 26a is not rotated but separated and shifted as desired in the direction indicated by a double headed arrow a in FIG. 3 relative to the coupling member 25.

Reference numeral 27 designates a nut member threadedly engaged with the screw portion 25a. This nut member 27 is pressed and the coupling member 25 is rotated so that the screw portion 25a is threadedly engaged with the screw hole 15. Reference numeral 28 designates a rotation preventing member of the double nut structure for preventing the rotation and shift of the shifting member 26 relative to the coupling member 25.

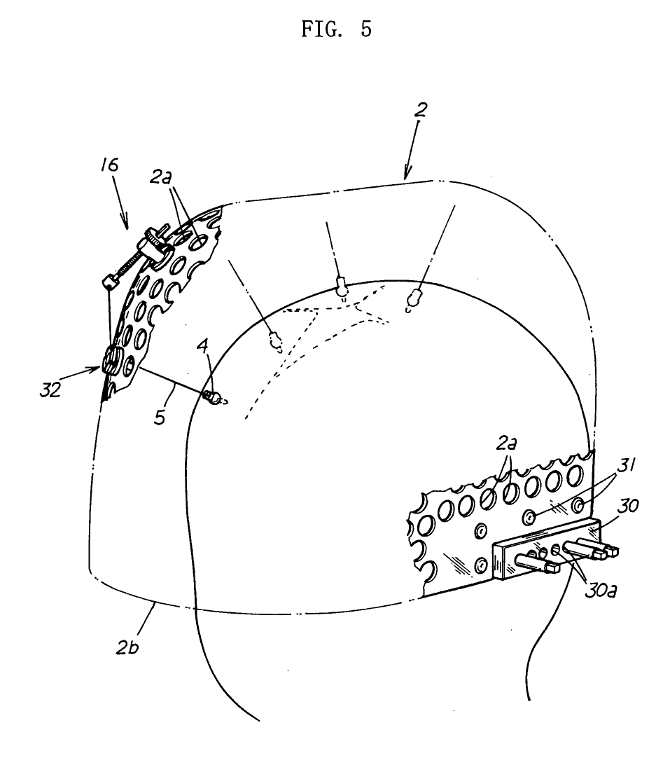

Also, it is possible to use a head portion arrangement body 2 as the type shown in FIG. 5 as the head portion arrangement body 2.

More specifically, the head portion arrangement body 2 is obtained by forming a suitable synthetic resin material into a curved shape (cage-like or helmet-like shape) shown in FIG. 5.

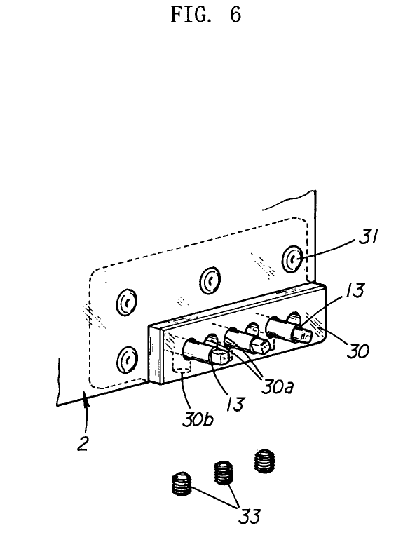

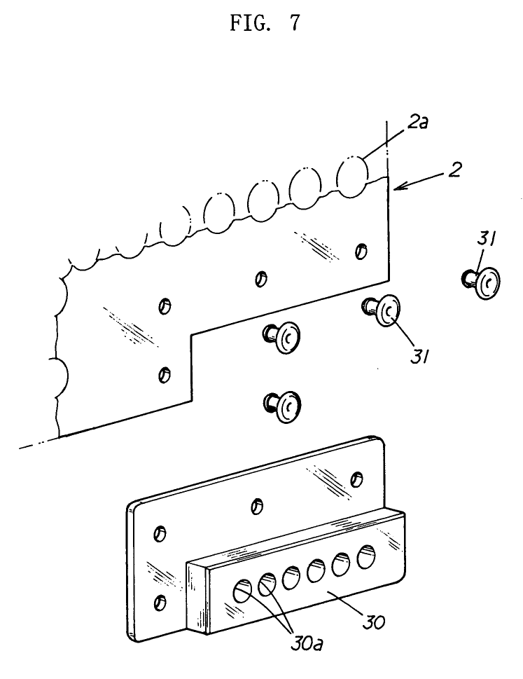

As shown in FIGS. 6 and 7, rod portion arrangement bodies 30 in which a plurality (six) of holes 30a are arranged in parallel in the lower rear portion of the head portion arrangement body 2 are retained on the right and left sides of the head portion arrangement body 2 by rivets 31. The holes 30a provided in each rod portion arrangement body 30 are adapted to serve as insertion holes 30a through which the fastening rod members 13 for fixing the head portion arrangement body 2 to the head 1 are inserted.

Incidentally, the fastening rod member 13 inserted into the insertion hole 30a of the rod portion arrangement body 30 is retained by the pressing contact of a screw 33 which passes through the through hole 30a from the lower surface of the rod portion arrangement body 30 and is screwed to the screw hole 30b.

The fastening rod member 13 is obtained by forming a suitable metal material into a rod shape. The screw portion 13a to be threadedly engaged with a proximal end portion of the retainer member 14 to be retained on the side portion of the cranium to be described later is projectingly provided to the tip end portion of the fastening rod member 13. The latter is threadedly attached to the retainer member 14 under the condition that the fastening rod member 13 is inserted through the insertion hole 30a of the rod member arrangement body 30 provided in the head portion arrangement body 2 and the head portion arrangement body 2 fitted to the head 1 at a predetermined interval is fixed to the head 1.

Incidentally, the retainer member 14 is retained at a retainer hole 3a bored in the cranium by using the retainer portion forming tool 18 and the boring tool 21 to be described later.

Also, as shown in FIG. 5, the head portion arrangement body 2 has a number of through holes 2a on its surface. In the case shown in the drawing, each of the through holes 2a is formed into a circular shape. It is possible to extract the line-like members 5 extending from the retainer members 4 to be described later through the through holes 2a to the outside of the head portion arrangement body 2.

It is possible to take any size of the through holes 2a but it is better to take a large size of the through holes 2a as much as possible in view of a light weight, permeability and an easy-to-handle property of the line-like members 5. Furthermore, it is preferable to take the triangular or the rectangular shape for the shape of the through hole 2a.

Also, the through hole 2a is adapted to serve as a retainer hole for detachably retaining the tension maintenance member 16 and a fixing member 32 to be described later.

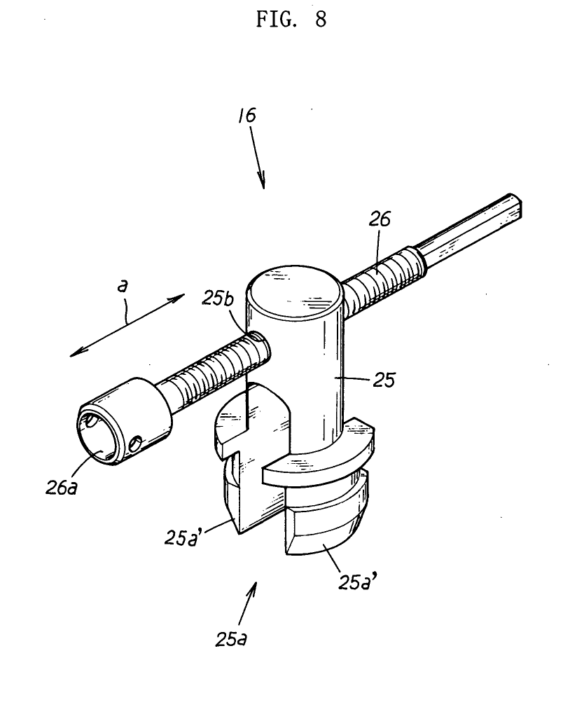

As shown in FIG. 8, the tension maintenance member 16 is made of a suitable synthetic resin material and is composed of a coupling member 25 provided with a fitting portion 25a retained to the through hole 2a provided in the lower portion of the head portion arrangement body 2 as described above and a shifting member 26 threadedly engaged with the screw hole 25b passing through in the perpendicular direction to the coupling member 25.

A pair of retainer pieces 25a′ are formed at the end portion of the coupling member 25. The retainer pieces 25a′ are made of synthetic resin and hence have the suitable flexibility. Thus, the pressing insertion and retention to the through hole 2a provided in the head portion arrangement body 2 of the coupling body 25 may be performed well. Accordingly, the tension maintenance member 16 may be detachably attached at any desirable position of the head portion arrangement body 2.

In the shifting member 26, a sleeve-like member 26a is rotatably fitted at the front tip portion thereof. This sleeve-like member 26a is formed into the coupling portion 26a for coupling with the line-like member 5 (wire) coupled to the retainer member 4 to be described later.

Accordingly, when the shifting member 26 is threadedly moved, the coupling portion 26a is not rotated but separated and shifted as desired in the direction indicated by a double headed arrow a in FIG. 8 relative to the coupling member 25.

Also, in this embodiment, there is provided a fastening member 32 detachably attached to the head portion arrangement body 2 for fastening the line-like member 5 removed from the tension maintenance member 16 after the completion of the tensed condition of the line-like member 5 by the above-described tension maintenance member 16.

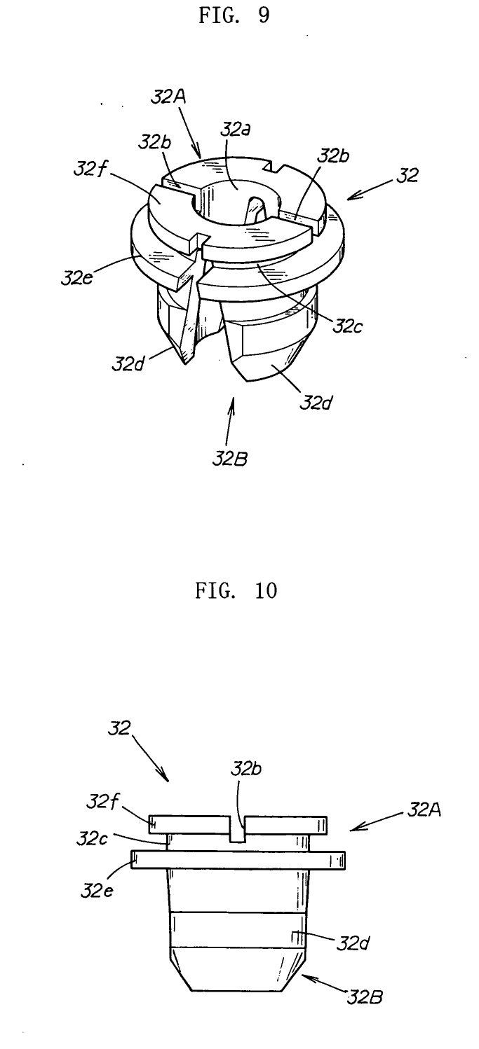

As shown in FIGS. 9 and 10, the fastening member 32 is formed of a suitable synthetic resin material (softer than that of the line-like member 5). The proximal end portion 32A is provided with a retainer portion 32B for being pressingly inserted into, retained at and fixed to the through hole 2a provided in the head portion arrangement body 2.

Also, in the fastening member 32, a through hole 32a is formed in its axial portion which is adapted to serve as a hole portion 32a for allowing the passage of the line-like member 5 extending from the retainer member 4.

Accordingly, this fastening member exhibits a function as a protecting member for preventing a damage of the through hole 2a provided in the head portion arrangement body 2 as much as possible by using it as a receiver of the line-like member 5 for an extension work period.

The proximal end portion 32A of the fastening member 32 is provided with a small diameter flange portion 32f through a small diameter barrel portion 32c on a large diameter flange portion 32e. Two concave grooves 32b are formed at an interval of 180° on this large diameter flange portion. The concave grooves 32b and a small diameter barrel portion 32c are portions for winding and retaining the line-like member 5 passing through the hole portion 32a. Furthermore, the concave grooves 32b exhibit a function to prevent a displacement of the line-like member 5 kept under the condition the line-like member 5 is caused to pass through the hole portion 32a and coupled with the tension maintenance member 16.

Also, the upper surface of the large diameter flange portion 32e is made flat. When the total height of the large diameter flange portion 32e, the small diameter barrel portion 32c and the small diameter flange portion 32f, i.e., the fastening member 32 is arranged in the head portion arrangement body 2, the height projecting from the surface of the head portion arrangement body 2 is set to be small as much as possible (at least smaller that an amount projecting from the surface of the head portion arrangement body 2 of the tension maintenance body 16).

As shown in FIGS. 9 and 10, the retainer portion 32B of the fastening member 32 has a structure in which a pair of retainer pieces 32d are formed at the end portion of the proximal end portion 32A thereof. The retainer pieces 32d have a suitable elasticity because the pieces are made of synthetic resin. The pressing insertion and retention of the fastening member 32 to the through hole 2a provided in the head portion arrangement body 2 may be performed well.

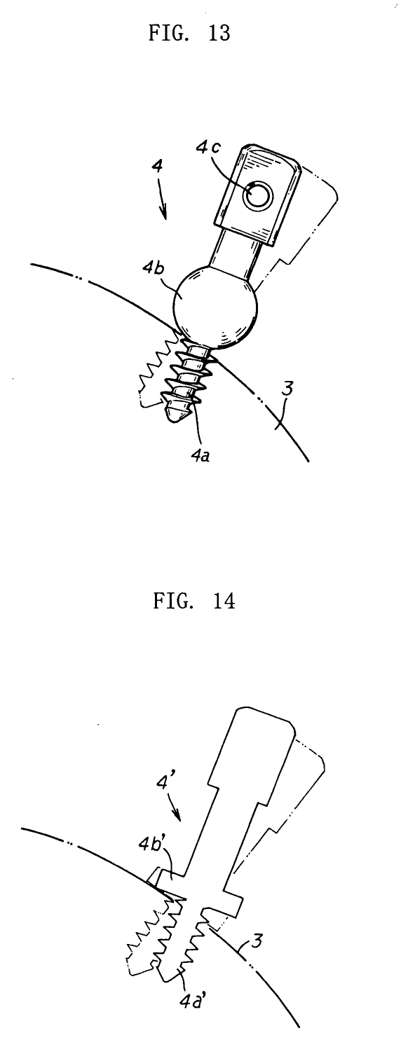

As shown in FIG. 13, the retainer member 4 is made of a suitable metal member. A screw-like insertion portion 4a is formed at its tip end portion. This insertion portion 4a is adapted to be screwed into a retainer hole 3a formed in the cranial piece 3.

Also, the retainer member 4 is provided in the proximal end portion of the insertion portion 4a with a large diameter portion 4b which is formed in a spherical shape and having a larger diameter than that of the insertion portion 4a for preventing the insertion portion 4a from entering into the cranial piece 3. Accordingly, the contact surface which is a lower surface of the large diameter portion 4b and is to be brought into contact with the cranial piece 3 becomes a curved surface.

As shown in FIG. 14, in the case where the large diameter proximal end portion 4b′ of the insertion portion 4a′ is in the form of a flange, even if the retainer member 4′ is screwed in a slanted condition to the cranial piece 3 slightly, the condition happens in which the insertion portion 4a′ is not sufficiently screwed into the cranial piece 3. However, in this respect, if the structure proposed by this embodiment, the insertion portion 4a is positively threadedly inserted into the cranial piece 3 while exhibiting its inherent function (suitable insertion preventing function). (The two-dot and dash lines in FIGS. 13 and 14 indicate the condition that the retainer members 4 and 4′ are threadedly inserted into the cranial piece 3 straightly.)

Also, the spherical shape of the large diameter portion 4b is effective also in the case where the retainer member 4 is removed away from the head 1 after the treatment.

Namely, as shown in FIG. 14, in the case where the large diameter proximal end portion 4b′ of the insertion portion 4a′ is in the form of a flange, the top surface of the large diameter portion 4b′ hooks at the skin. Accordingly, it is necessary to cut the skin when the retainer member 4 from the head 1. However, in this respect, in this embodiment, since the large diameter portion 4b is spherical, there is no fear that the large diameter portion 4b would hook at the skin. It is therefore unnecessary to open the skin and it is possible to remove the retainer member 4 from the cranial piece 3 of the head 1 without damaging the skin as much as possible. (It is possible to reduce the damage to be imposed on the patient.)

Also, the hole 4c is formed in the proximal end portion of the retainer member 4. The hole 4c is formed as a coupling hole 4c for coupling the line-like member 5 (wire).

Incidentally, the wire is used as the line-like member 5. However, this line-like member 5 is not limited to the string-like one but includes a concept in a wide range such as a rod-like one.

In the case where this retainer member 4 is to be retained at the cranial piece 3, the boring tool 21 is used to form the retainer hole 3a in the cranial piece 3 and the retainer member 4 is retained at this retainer hole 3a.

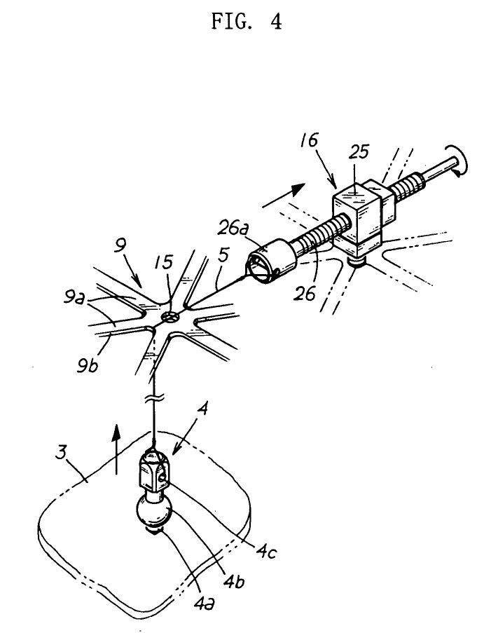

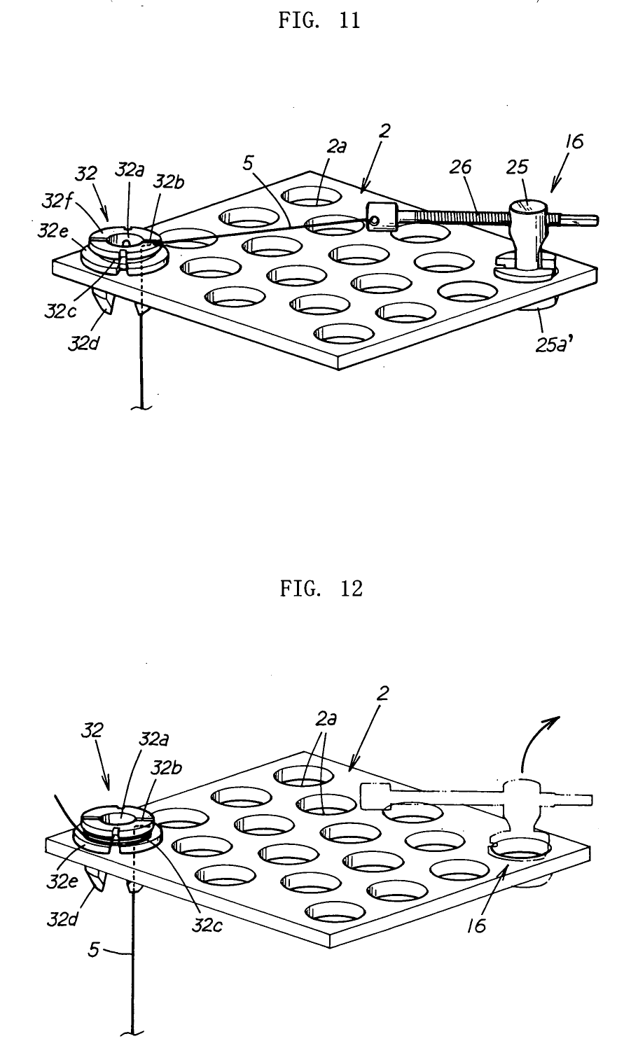

With such a structure, as shown in FIGS. 4 and 11, when the shifting member 16B of the tension maintenance body 16 coupled to the head portion arrangement body 2 (which head portion arrangement body 2 is shown in a square-shape in FIG. 11 for the sake of explanation) is threadedly moved to move backward, the line-like member 5 coupled with the coupling hole 4c of the retainer member 4 is tensed through the fastening member 32 (hole portion 32a and concave groove 32b) retained at an opening edge (through hole 2a) of the through hole 9a of the head portion arrangement body 2 located above the retainer member 4 so that the retainer member 4 is tensed and the cranial piece 3 is tensed upwardly (in the radial direction of the head 1). When the movement of the tension maintenance member 16 is stopped, the tensed condition of the cranial piece 3 may be maintained.

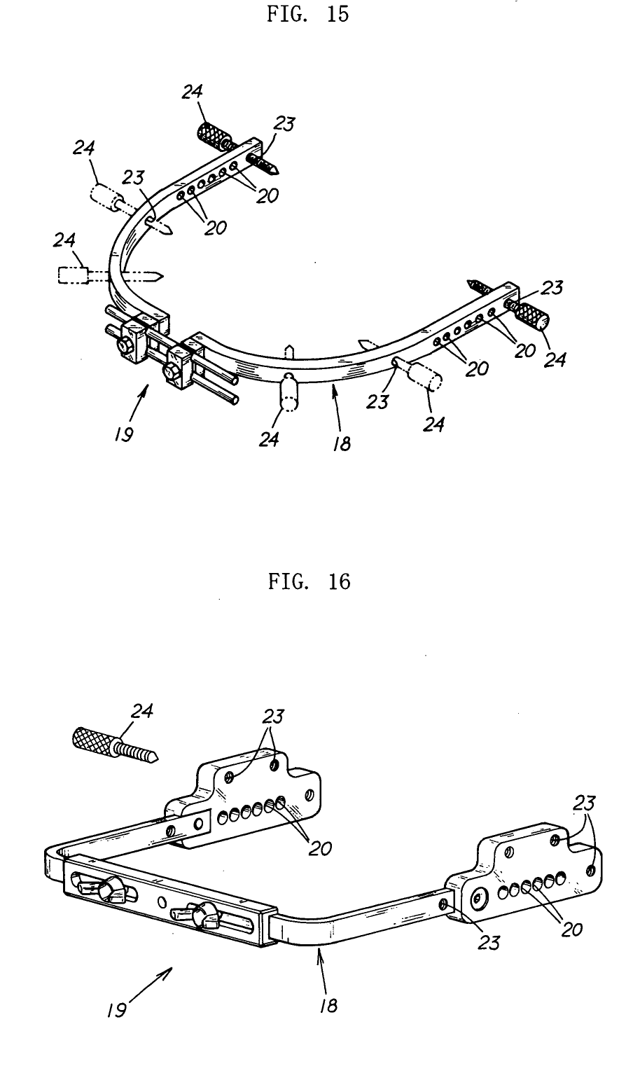

As shown in FIGS. 15 and 16, the retainer portion forming tool 18 is obtained by forming a suitable metal material. The tool is a U-shaped frame member as viewed in plan view and divided in right and left halves and has a shape identified with the lower end portion of the head portion arrangement body 2.

Also, the size of the retainer portion forming tool 18 may be set in accordance with a size of the head 1 by adjusting an interval adjustment mechanism 19 provided at its end portions.

Also, in the retainer portion forming tool 18, six holes 20 are formed in parallel on each side of linear portions of the right and left sides. The holes 20 are formed at positions identified with fitting holes 12 provided in the above-described head portion arrangement body 2. The retainer portion forming tool 18 exhibits the function as a guide hole for guiding a rotary tool 22 for rotationally retaining the retainer member 14 and the boring tool 21 for forming the retainer hole 3a at which the retainer member 14 is retained at the cranial piece 3.

Also, holes 23 are formed at a predetermined interval in the peripheral portion of the retainer portion forming tool 18. The holes 23 exhibit the function as insertion holes through which fastening rods 24 are fitted for fixing to the head 1 the retainer portion forming tool 18 under the condition that the retainer portion forming tool 18 is arranged around the head 1.

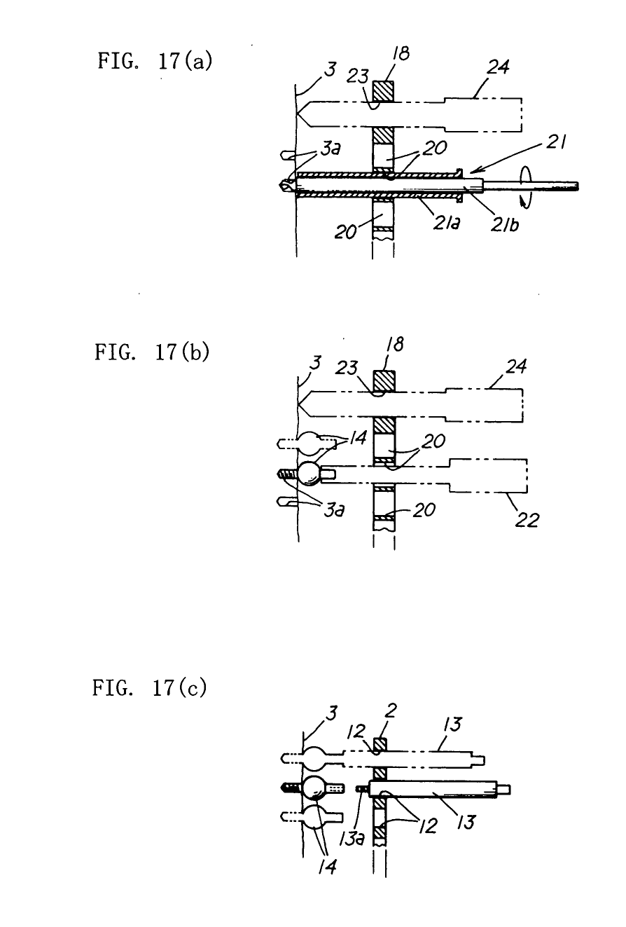

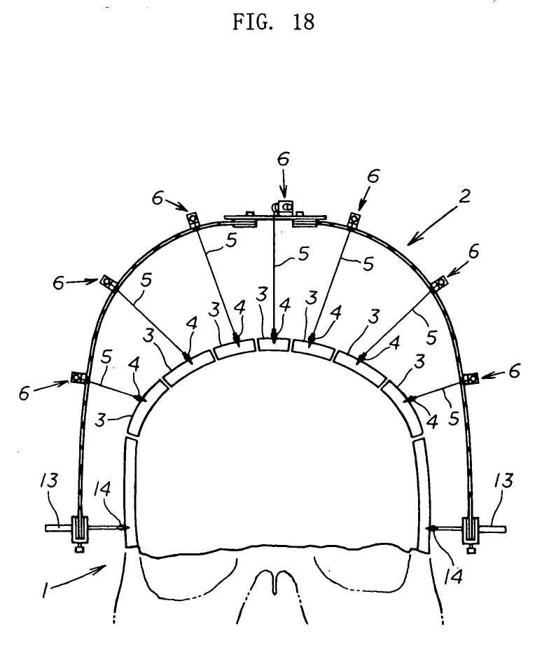

More specifically, describing how to retain the retainer member 14 to the cranial piece 3 using the retainer portion forming tool 18, as shown in FIG. 17, the retainer portion forming tool 18 is arranged around the head 1 and the fixing rods 24 are caused to pass through the holes 23. The fixing rods 24 are retained at the side portion of the cranium and the retainer portion forming tool 18 is fixed in a predetermined position. Under this condition, the boring tool 21 composed of a guide sleeve member 21a and a drill 21b is inserted into the hole 20 to form the retainer hole 3a in a predetermined position of the cranial piece 3 (see FIG. 17(a)). The rotary tool 22 is used for the retainer hole 3a and the retainer member 14 is rotationally retained (see FIG. 17(b)). Thereafter, the fastening rod member 13 coupled with the above-described head portion arrangement body 2 is coupled with the retainer member 14 retained at the cranial piece 3 (see FIG. 17(c)).

Incidentally, the retainer member 14 has substantially the same structure as the above-described retainer member 4. A screw hole for threadedly engaging a screw portion 13a projecting at the tip end portion of the fastening rod member 13 is formed in the proximal end portion of the retainer member 14.

A treatment of the cranium early-stage agglutination using the structure according to the foregoing embodiment will now be described. This embodiment is not limited to the cranium early-stage agglutination but may be extensively applied to a cranium treatment (bone extension method) such as a treatment for a malformation or a break of a cranium.

First of all, the head skin is cut and opened to divide the cranial pieces 3. The retainer member 4 provided with the line-like member 5 is retained at each cranial piece 3. When this retainer member 4 is to be retained, as described above, the retainer member 4 is retained at the retainer hole 3a formed in advance in the cranial piece 3 by the boring tool 21.

Subsequently, the retainer member 14 is retained at the side portion of the cranial piece 3 by using the retainer portion forming tool 18. The fastening rod member 13 is coupled to the retainer member 14 in the fitted condition of the head portion arrangement body 2 to the head 1 to thereby fix the head portion arrangement body 2 in place. In the case of the head portion arrangement body 2 of the type shown in FIG. 1, the retainer member 14 is retained at the side portion of the cranial piece 3 by using the retainer member forming tool 18. The fastening rod member 13 is coupled with the retainer member 14. The halves 2A and 2B constituting the head portion arrangement body 2 are arranged from the right and left sides while inserting the fastening rod members 13 into the insertion holes 12 and the halves 2A and 2B are coupled with each other so that the head portion arrangement body 2 may be fitted around the head 1.

Subsequently, the line-like member 5 coupled with the retainer member 4 is extracted to the outside through the through hole 9b (the fixing member 32 retained at the through hole 2a) of the head portion arrangement body 2 located just above the retainer member 4. The extracted portion is coupled with the coupling portion 26a of the shifting member 26 of the tension maintenance member 16 retained at the head portion arrangement body 2. Incidentally, the stage when the line-like member 5 is coupled with the tension maintenance member 16 may be before the tension maintenance member 16 is coupled with the head portion arrangement body 2.

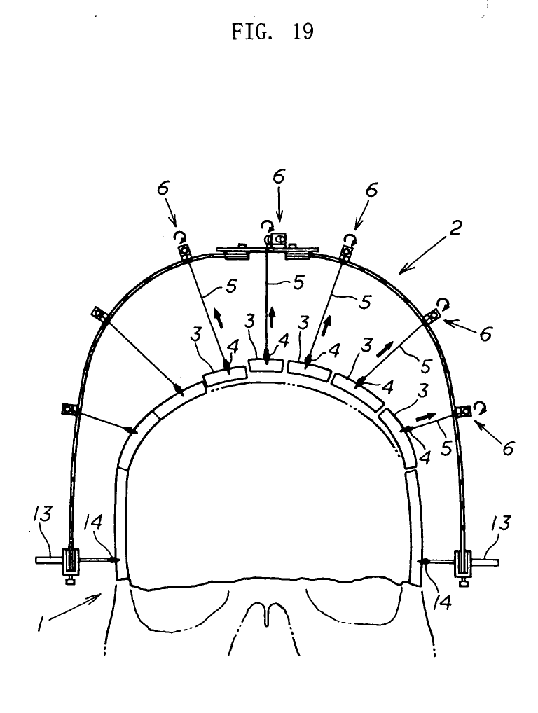

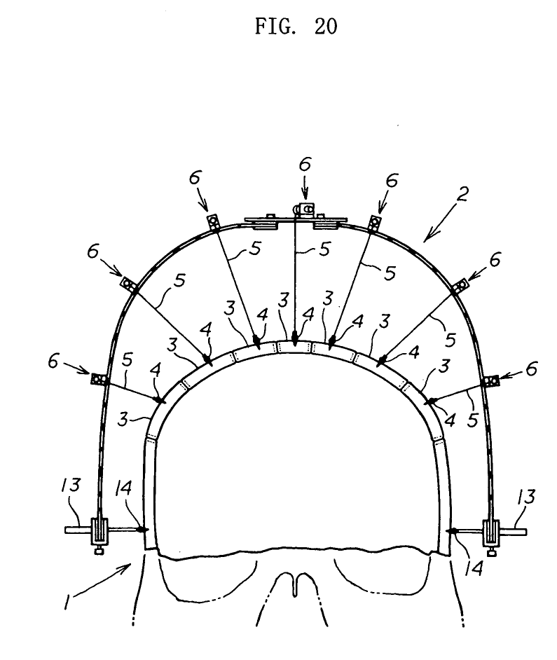

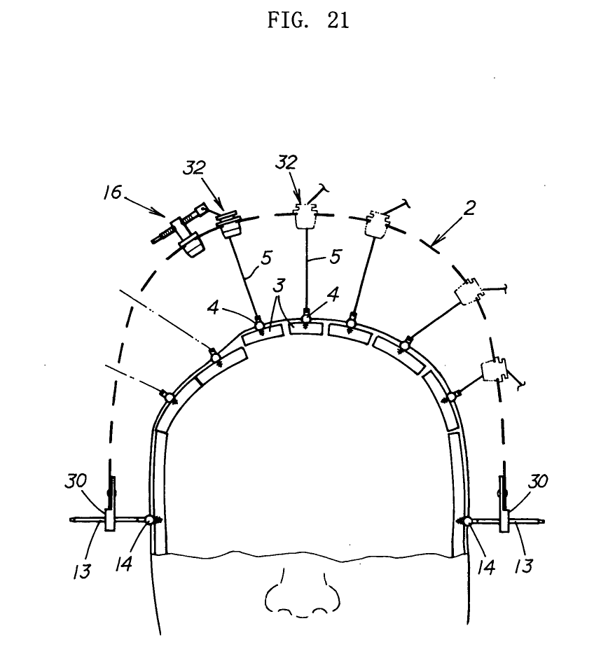

Subsequently, each tension maintenance member 16 set as described above is operated to tense the retainer member 4 upwardly and each cranial piece 3 is moved in the radial direction of the head 1 while separating the cranial pieces 3 away from each other. (Each cranial piece 3 is tensed in the radial direction, and the cranial pieces 3 are moved in the three-dimensional directions while separating away from each other so that a so-called spherical diameter is increased.) This condition may be maintained (see FIGS. 18, 19 and 21). FIG. 20 shows a condition (in which the treatment is completed) in which the cranial pieces 3 that have been separated from each other by using the apparatus according to the embodiment are extended to be agglutinated with each other. This is the extension work stage for the disease of the cranium early-stage agglutination.

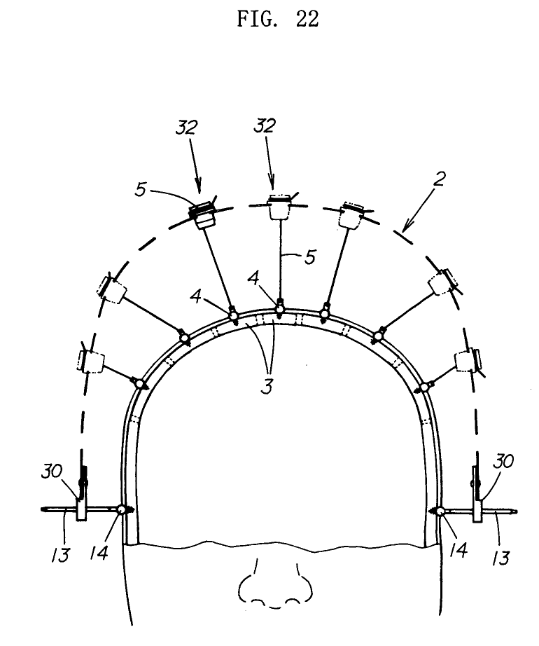

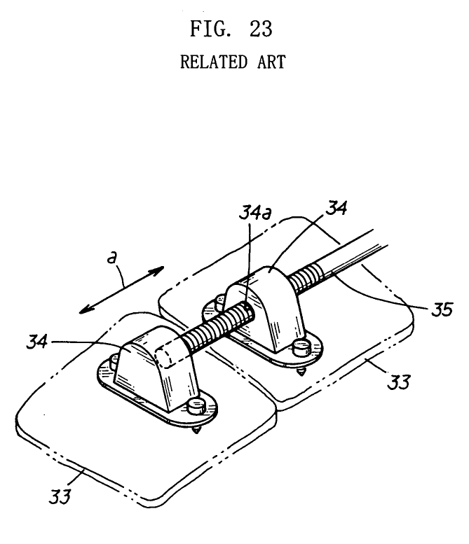

Subsequently, after the extension work stage has been ended, the tensed condition of the line-like member 5 by the tension maintenance member 16 is completed. The line-like member 5 that has been removed from the tension maintenance member 16 is fixed to the fastening member 32 to thereby maintain this condition until the agglutination of the cranial pieces 3 to each other is completed without the cranial pieces 3 being returned back to the original condition (see FIG. 22). This is the fixing work stage of the treatment for the cranium early-stage agglutination.

Since the present embodiment has been constructed as described above, the condition in which each cranial piece 3 is tensed in the radial direction of the head 1, i.e., the direction cranial pieces 3 are separated away from each other (radial direction of the head 1) may be maintained to thereby make it possible to prevent the agglutination of the cranial pieces 3 with each other. Furthermore, since the cranial pieces 3 are not tensed in the two-dimensional direction as in the related art but may be tensed in the three-dimensional direction, the cranial pieces 3 complies with the direction of development of the brain (i.e., swelling direction). Accordingly, it is possible to keep the shape of the cranial pieces 3 in a very good condition.

Also, in this embodiment, after the completion of the tensed condition of the line-like member 5 by the tension maintenance member 16, the fastening member 32 for fixing the line-like member 5 that has been removed from the tension maintenance member 16 may be detachably provided to the head portion arrangement body 2. Under the condition the fastening member 32 is arranged on the head portion arrangement body 2, the projecting amount from the surface of the head portion arrangement body 2 is set to be smaller than the projecting amount from the head portion arrangement body 2 of the above-described tension maintenance member 16. In the case where the cranial pieces 3 are not tensed, if the tension maintenance member 16 projecting from the head portion arrangement body 2 is removed away, there is no obstruction that projects from the head portion arrangement body 2 so that it is possible to perform the treatment to such an extent.

Also, in this embodiment, the fastening member 32 is detachably provided in the through hole 2a provided in the head portion arrangement body 2, and the retainer portion for retaining the line-like member 5 extending from the retainer member 4 is provided. Accordingly, it is possible to mount the fastening member 32 on the head portion arrangement body 2 when necessary and to fix the line-like member 5 in place.

Also, in this embodiment, since the fastening member 32 is made of softer material than that of the line-like member 5, it is possible to prevent the cut of the line-like member 5 as mush as possible.

Also, in this embodiment, since the fastening member 32 takes a structure in which the line-like member 5 is wound and fixed, the hard fixture may readily be performed without fail. In addition, since there is no possibility that the line-like member 5 for being fixed to the fastening member 32 is arranged above the topmost surface of the fastening. Accordingly, it is possible to suppress the fear that unnecessary projection from the head portion arrangement body 2 would be generated.

Also, in this embodiment, since the fastening member 32 is provided with the hole 32a for allowing the line-like member 5 extending from the retainer member 4 to pass therethrough, the fastening member 32 is used as a portion through which the line-like member 5 is caused to pass even in the extension work period of the tension maintenance member 16. Accordingly, there is no fear that the line-like member 5 is brought into direct contact with the head portion arrangement body 2. It is therefore possible to prevent a damage of the head portion arrangement body 2 made of synthetic resin as much as possible.

Incidentally, the present invention is not limited to the specific embodiments. The specific structure of each structural element is subject to change as necessary.

1. A cranial treatment apparatus comprising:
a head portion arrangement body for arranging around a head; a retainer member retained at each cranial piece, being tensed by a tension maintenance member for maintaining a tensed condition so that cranial pieces are tensed in a direction away from each other, said retainer member being provided in the head portion arrangement body.

2. The cranial treatment apparatus according to claim 1, wherein the direction in which said tension maintenance member tenses is in a radial direction of the head.

3. The cranial treatment apparatus according to claim 1, wherein said tension maintenance member is adapted to tense the retainer member and maintain the tensed condition.

4. The cranial treatment apparatus according to claim 2, wherein said tension maintenance member is adapted to tense the retainer member and maintain the tensed condition.

5. The cranial treatment apparatus according to claim 1, wherein said retainer member and said tension maintenance member are coupled with each other by a line-like member so that the retainer member is tensed by the line-like member and the tensed condition is kept by the tension maintenance member.

6. The cranial treatment apparatus according to claim 5, wherein a fastening member is detachably provided in said head portion arrangement body for fixing the line-like member that has been removed from the tension maintenance member after the tensed condition completion of the line-like member by said tension maintenance member, and a projecting amount from a surface of said head portion arrangement body of said fastening member is set to be smaller than a projecting amount from a surface of the head portion arrangement body of said tension maintenance member.

7. The cranial treatment apparatus according to claim 6, wherein a hole portion for allowing the line-like member extending from the retainer member to pass therethrough is provided in said fastening member.

8. The cranial treatment apparatus according to claim 1, wherein said retainer member has an insertion portion which is inserted into the cranial piece, a large diameter portion which is larger in diameter than the insertion portion and prevents the insertion portion from entering into the cranial piece is formed on a proximal end side of the insertion portion, and a lower surface or of the side of the insertion portion of the large diameter portion is formed into a curved surface or a slant surface.

9. The cranial treatment apparatus according to claim 5, wherein said retainer member has an insertion portion to be inserted into the cranial piece, a large diameter portion that is larger in diameter than the insertion portion for preventing the insertion portion from entering into the cranial piece is formed on the proximal end side of the insertion portion, and the lower surface on the insertion portion side of the large diameter portion is formed into a curved surface or a slant surface.

10. The cranial treatment apparatus according to claim 6, wherein said retainer member has an insertion portion to be inserted into the cranial piece, a large diameter portion that is larger in diameter than the insertion portion for preventing the insertion portion from entering into the cranial piece is formed on the proximal end side of the insertion portion, and the lower surface on the insertion portion side of the large diameter portion is formed into a curved surface or a slant surface.

11. The cranial treatment apparatus according to claim 7, wherein said retainer member has an insertion portion to be inserted into the cranial piece, a large diameter portion that is larger in diameter than the insertion portion for preventing the insertion portion from entering into the cranial piece is formed on the proximal end side of the insertion portion, and the lower surface on the insertion portion side of the large diameter portion is formed into a curved surface or a slant surface.
